(19) United States
(12) Patent Application Publication
(10) Pub. No.: US 2024/0358412 A1
(43) Pub. Date: Oct. 31, 2024

(54) ADJUSTABLE IMPLANT, SYSTEM AND METHODS

(71) Applicant: NUVASIVE SPECIALIZED ORTHOPEDICS, INC., San Diego, CA (US)

(72) Inventor: Jorge Lopez Camacho, Oxnard, CA (US)

(21) Appl. No.: 18/573,840

(22) PCT Filed: Jun. 17, 2022
(86) PCT No.: PCT/US2022/033977

§ 371 Date: Jan. 10, 2024

Related U.S. Application Data
(60) Provisional application No. 63/215,105, filed on Jun. 25, 2021.

(51) Int. Cl. A61B 17/70; A61B 17/00; A61B 17/68; A61B 17/72

(52) U.S. Cl. CPC A61B 17/7016; A61B 17/7216; A61B 2017/00199; A61B 2017/681

(57) ABSTRACT

One aspect of the disclosure relates to an adjustable implant. The adjustable implant may include a housing configured to be coupled to a first bone portion; an adjustable portion configured to be coupled to a second bone portion, the adjustable portion having a first bar; an actuator rotationally mounted within the housing, the actuator including a protrusion extending therefrom; and at least one gear having an anvil coupled thereto, wherein the protrusion of the actuator is configured to engage the anvil during rotation of the actuator to cause the adjustable portion to move relative to the housing. The protrusion may include an impact hammer surface. Also provided herein are distraction and compression systems including adjustable implants and adjustment devices therefor, and methods for adjusting such adjustable implants.

### CROSS REFERENCE TO RELATED APPLICATIONS

The present patent application claims priority to International Application No. PCT/US22/33977, files Jun. 17, 2022, which claims priority to U.S. Provisional Application No. 63/215,105, filed Jun. 25, 2021, the entirety of both of which are incorporated by reference as though fully set forth herein.

### TECHNICAL FIELD

The subject matter described herein relates to an adjustable implant, system and associated methods.

### BACKGROUND

Distraction osteogenesis is a technique which has been used to grow new bone in patients with a variety of defects. For example, limb lengthening is a technique in which the length of a bone (for example a femur or tibia) may be increased. By creating a corticotomy, or osteotomy, in the bone, which is a cut through the bone, the two resulting sections of bone may be moved apart at a particular rate, such as one (1.0) mm per day, allowing new bone to regenerate between the two sections as they move apart. This technique of limb lengthening is used in cases where one limb is longer than the other, such as in a patient whose prior bone break did not heal correctly, or in a patient whose growth plate was diseased or damaged prior to maturity. In some patients, stature lengthening is desired, and is achieved by lengthening both femurs and/or both tibias to increase the patient's height.

Limb lengthening is often performed using external fixation, wherein an external distraction frame is attached to the two sections of bone by pins which pass through the skin. The pins can be sites for infection and are often painful for the patient, as the pin placement or “pin tract” site remains a somewhat open wound throughout the treatment process. The external fixation frames are also bulky, making it difficult for patient to comfortably sit, sleep and move. Intramedullary lengthening devices also exist, such as those described in U.S. Patent Application Publication No. 2011/0060336, which is incorporated by reference herein.

### SUMMARY

A first aspect of the disclosure relates to an adjustable implant. The adjustable implant may include: a housing configured to be coupled to a first bone portion; an adjustable portion configured to be coupled to a second bone portion, the adjustable portion having a first bar; an actuator rotationally mounted within the housing, the actuator including a protrusion extending therefrom; and at least one gear having an anvil coupled thereto, wherein the protrusion of the actuator is configured to engage the anvil during rotation of the actuator to cause the adjustable portion to move relative to the housing.

A second aspect of the disclosure relates to an adjustable implant. The adjustable implant may include: a housing configured to be coupled to a first bone portion; an adjustable portion configured to be coupled to a second bone portion, the adjustable portion having a toothed rack; a magnet assembly rotationally mounted within the housing, the magnet assembly including a protrusion extending therefrom, the protrusion having an impact hammer surface; and at least one gear having an anvil coupled thereto, wherein the protrusion of the magnet assembly is configured to engage the anvil during rotation of the magnet assembly, whereby such engagement of the anvil by the impact hammer surface of the protrusion causes the at least one gear to interact with the toothed rack thereby causing the adjustable portion to move relative to the housing.

A third aspect of the disclosure relates to an adjustable implant. The adjustable implant may include: a housing configured to be coupled to a first bone portion; an adjustable portion configured to be coupled to a second bone portion, the adjustable portion having a toothed rack; a magnet assembly rotationally mounted within the housing, the magnet assembly including a protrusion extending therefrom; a first gear having a first anvil coupled thereto; and a second gear having a second anvil coupled thereto, wherein the protrusion of the magnet assembly is configured to engage the first and second anvils during rotation of the magnet assembly, whereby such engagement of the first and second anvils by the protrusion causes the first and second gears to interact with the toothed rack thereby causing the adjustable portion to move relative to the housing.

A fourth aspect of the disclosure relates to a method of non-invasively adjusting an adjustable implant. The method includes: providing an adjustable implant, the adjustable implant including a housing configured to be coupled to a first bone portion; an adjustable portion configured to be coupled to a second bone portion, the adjustable portion having a first bar; an actuator rotationally mounted within the housing, the actuator including a protrusion extending therefrom; and at least one gear having an anvil coupled thereto; coupling the housing to the first bone portion; coupling the adjustable portion to the second bone portion; and non-invasively adjusting the adjustable implant by causing rotation of the actuator such that the protrusion of the actuator engages with the anvil during rotation of the actuator to cause the adjustable portion to move relative to the housing.

A fifth aspect of the disclosure relates to a system. The system includes: an adjustable implant including: a housing configured to be coupled to a first bone portion; an adjustable portion configured to be coupled to a second bone portion, the adjustable portion having a first bar; an actuator rotationally mounted within the housing, the actuator including a protrusion extending therefrom; and at least one gear having an anvil coupled thereto, wherein the protrusion of the actuator is configured to engage the anvil during rotation of the actuator to cause the adjustable portion to move relative to the housing; and an external adjustment device configured to cause actuation of the actuator upon being positioned such that the external adjustment device is perpendicular to the actuator.

A sixth aspect of the disclosure relates to an assembly for an adjustable implant. The assembly includes: an actuator configured to be rotatably mounted within the adjustable implant, the actuator including a protrusion extending therefrom; a first gear having a first anvil coupled thereto; and a second gear having a second anvil coupled thereto, wherein rotation of the actuator causes the protrusion to engage the first anvil and the second anvil to cause the adjustable implant to adjust.

### BRIEF DESCRIPTION OF DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 shows a perspective view of an adjustable implant according to embodiments of the disclosure;

FIG. 2 shows a perspective cross-sectional view of the adjustable implant of FIG. 1;

FIG. 3 shows a perspective view of the actuator according to embodiments of the disclosure;

FIG. 4 shows a cross-sectional view of the adjustable implant in a fully retracted position, according to an embodiment of the disclosure;

FIG. 5 shows a cross-sectional view of the adjustable implant in a first distracted position, according to an embodiment of the disclosure;

FIG. 6 shows a cross-sectional view of the adjustable implant in a second distracted position, according to an embodiment of the disclosure;

FIG. 7 shows a perspective view of a system for non-invasively adjusting a first bone portion and a second bone portion according to embodiments of the disclosure;

FIG. 8 shows a front view of an external adjustment device according to embodiments of the disclosure;

FIG. 9 shows a cross-sectional side view of the external adjustment device of FIG. 8, according to embodiments of the disclosure;

FIG. 10 shows a cross-sectional view of a magnet drive system including a motor having an internal motor speed sensor, according to embodiments of the disclosure;

FIG. 11A shows a magnet of an external adjustment device magnetically coupled to a permanent magnet of an adjustable implant, according to embodiments of the disclosure;

FIG. 11B shows a magnet of the external adjustment device magnetically coupled to the permanent magnet of the adjustable implant, according to embodiments of the disclosure;

FIG. 12 shows a perspective view of a system for non-invasively adjusting a first bone portion and a second bone portion according to aspects of the disclosure;

FIG. 13 shows an exploded view of the housing and adjustable portion of an adjustable implant according to another embodiment of the disclosure;

FIG. 14 shows a cross-sectional view of the adjustable implant of FIG. 13 in a fully retracted position, according to embodiments of the disclosure; and

FIG. 15 shows a cross-sectional view of the adjustable implant of FIG. 13 in a distracted position, according to embodiments of the disclosure.

It is noted that the drawings of the subject matter are not necessarily to scale. The drawings are intended to depict only typical aspects of the subject matter, and therefore, should not be considered as limiting the scope of the disclosed subject matter. In the drawings, like numbering represents like elements between the drawings.

### DETAILED DESCRIPTION

The present disclosure describes various embodiments of an adjustable implant, a system including an adjustable implant and an external adjustment device, and associated methods of adjusting the adjustable implant. The embodiments described herein can be used in the context of an extramedullary limb lengthening device and system or an intramedullary limb lengthening device and system. It is also contemplated that the embodiments described herein can be used in spinal fixation devices and systems, such as for example, in the treatment of scoliosis or in interspinous process devices. As is described herein, the adjustable implant is driven under impact instead of by a direct drive assembly, resulting in greater distraction for lengthening applications and/or greater retraction force for compression applications (e.g., non-union fractures).

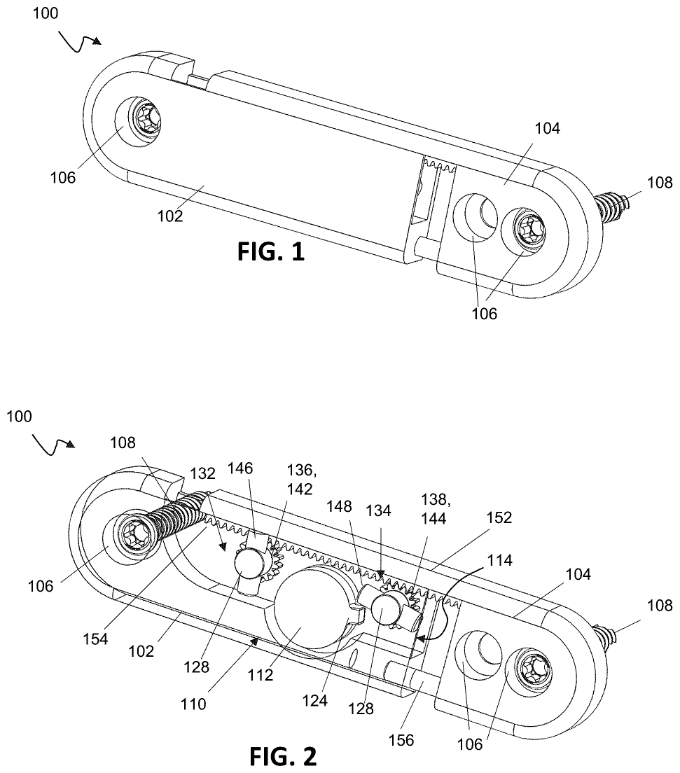

FIGS. 1 and 2 show an adjustable implant 100 according to aspects of the disclosure. The adjustable implant 100 includes a housing 102 configured to be coupled to a first bone portion and an adjustable portion 104 configured to be coupled to a second bone portion. Each of the housing 102 and the adjustable portion 104 can include one or more fixation apertures 106 therein. The fixation apertures 106 may be configured to receive a bone anchor 108 (e.g., a bone fastener, fastener, fixation screw, spinal fixation screw, bone screw, or pedicle screw) therein for securing the adjustable implant 104 to a bone (e.g., a femur, tibia, humerus, one or more pedicles or vertebrae, etc.). The adjustable portion 104 is configured to be moveable relative to the housing 102 to move the second bone portion relative to the first bone portion. Where the movement of the second bone portion is in a direction away from the first bone portion, osteogenesis may occur as will be discussed herein. It is to be understood that the outer profile and/or the overall dimensions (e.g., length, width, height, etc.) of the adjustable implant 100 can be customized based on the desired application of the adjustable implant 100 (i.e., limb lengthening, scoliosis treatment, etc.).

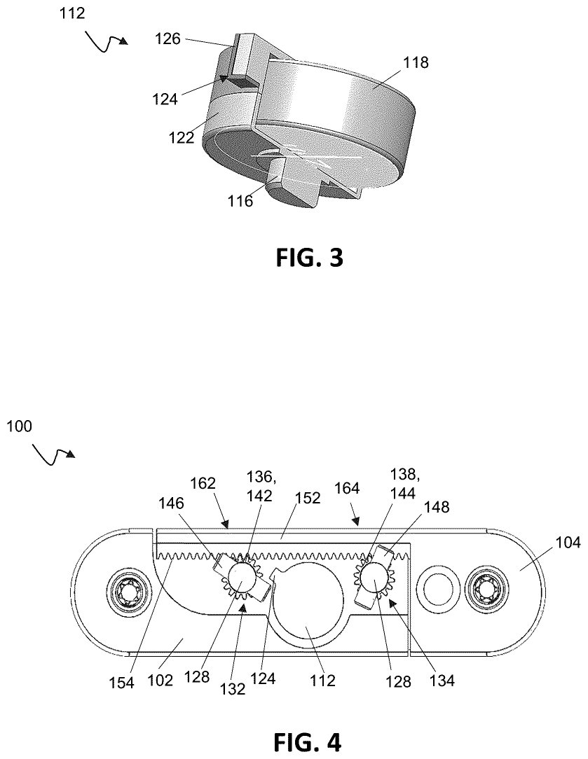

The adjustable implant 100 also includes an assembly 110 (see FIG. 2) for causing adjustment of the adjustable implant. The assembly 110 includes an actuator 112 rotationally mounted within a cavity 114 of the housing 102, for example, via a pin 116 (FIG. 3). However, any other now known or later developed means for rotationally mounting the actuator 112 within the housing 102 can also be used without departing from aspects of the disclosure, such as, for example, a knob, extension, tack, screw, fastener, and/or equivalents thereof. In some embodiments, the actuator 112 can include a stored energy spring, a pressure vessel with relief valve, or a small motor. As shown in FIG. 3, in some embodiments, the actuator 112 can include a magnet 118 and a magnet casing 122. The magnet 118 may include a cylindrical permanent magnet having a north and south pole. The actuator 112 includes a protrusion 124 extending radially therefrom. More specifically, the protrusion 124 can extend from the magnet casing 122. As used herein, “protrusion” may include a bump, knob, jut, projection, prominence, protuberance, overhang, ridge, ledge, and/or equivalents thereof. The protrusion 124 can be integrally formed with the actuator 112 (e.g., the magnet casing 122) and/or fixedly attached thereto. The protrusion 124 can include an impact hammer surface 126. As will be described in more detail herein, the impact hammer surface 126 acts to provide a large, striking force to cause translation of the adjustable portion 104 relative to the housing 102. The impact hammer surface 126 is composed of a material having sufficient strength and/or hardness to act as an impact hammer without softening the blow each time the impact hammer surface 126 strikes. Examples of such materials include, but are not limited to, chrome plating, nitride-treated steels 64-70 on the Rockwell scale (Rc), plasma nitriding, case hardening, and diamond-like coatings (DLC). In other embodiments, the protrusion 124 may include an amorphous metal alloy (liquid metal) insert.

Further, the assembly 110 of the adjustable implant 100 also includes at least one gear assembly rotationally mounted within the cavity 114 of the housing 102, for example, via a pin 128. However, any other now known or later developed means for rotationally mounting the gear assembly within the housing 102 can also be used without departing from aspects of the disclosure, such as, for example, a knob, extension, tack, screw, fastener, and/or equivalents thereof. As shown in FIG. 2, the assembly 110 for the adjustable implant 100 can include two gear assemblies, a first gear assembly 132 and a second gear assembly 134. The two gear assemblies 132, 134 may be positioned on opposing sides of the actuator 112 within the cavity 114. Each gear assembly 132, 134 can include a gear 136, 138 (e.g., a pinion gear) having gear teeth 142, 144 and an anvil 146, 148 coupled thereto. As shown, in some embodiments, the anvil 146, 148 of each gear assembly 132, 134 may extend at least partially through the pin 128, for example, through an aperture formed within the pin 128. However, it is to be understood that any other now known or later developed means for coupling the anvil 146, 148 to the gears 136, 138 can be used without departing from aspects of the disclosure, such as, for example, a tack, weld, bond, joint, and/or equivalents thereof.

As shown in FIGS. 1-2 and 4-6, the adjustable portion 104 includes at least one bar 152. The bar 152 can include, for example, a gear rack having a plurality of teeth 154 extending along a substantial length of the bar 152. However, other configurations are also contemplated by aspects of the disclosure. For example, the bar 152 can include a bar, rod, shaft, rail, or strut with ridges, teeth, threads, or bumps configured to mesh with the gears 136, 138. The bar 152 can be integrally formed with the adjustable portion 104 or fixedly attached thereto. The adjustable portion 104 can include an optional second bar 156 at least partially disposed within the housing 102 for providing additional strength and aid in guiding the movement of the adjustable portion 104 relative to the housing 102. The bar 156 can be configured to move relative to the housing 102 as the adjustable portion 104 moves relative to the housing 102. For example, the bar 156 can slide and/or translate within a second cavity (not shown) within the housing 102 during movement of the adjustable portion 104. The bar 156 can be integrally formed with the adjustable portion 104 or fixedly attached thereto. In other embodiments, the bar 156 can be replaced with male and female sleeves for providing added guidance and strength.

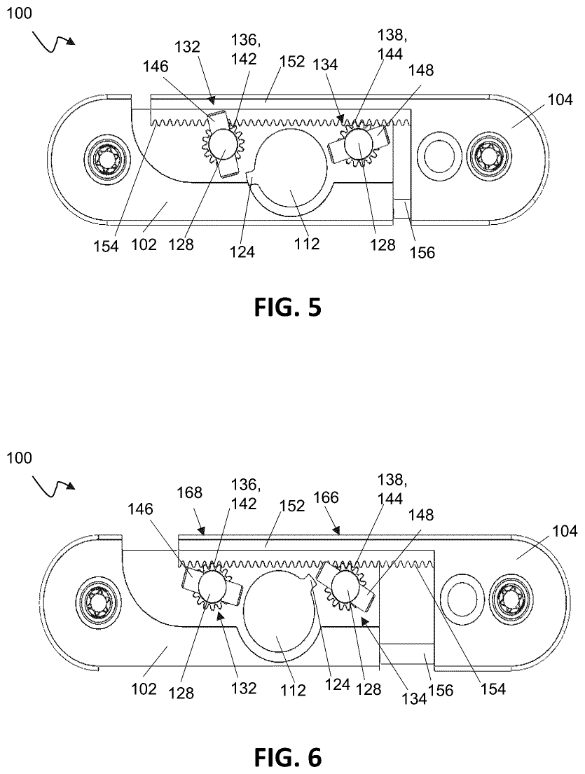

Turning now to FIGS. 4-6, operation of the adjustable implant 100 will now be described. FIG. 4 shows the adjustable implant 100 in a fully retracted state, FIG. 5 shows the adjustable implant 100 in a first distracted state after the anvil 146 has been engaged or stricken by the protrusion 124, and FIG. 6 shows the adjustable implant 100 in a second distracted state after anvil 148 has been engaged or stricken by the protrusion 124.

Upon actuation (i.e., rotation) of the actuator 112, the protrusion 124 of the actuator 112 is configured to engage with the anvil 146 of the first gear assembly 132. More specifically, upon actuation of the actuator 112, the impact hammer surface 126 (FIG. 3) of the protrusion 124 provides a striking force on the anvil 146 of the first gear assembly 132. Such engagement and/or striking force causes the gear 136 to rotate and the gear teeth 142 to mesh with the teeth 154 on the bar 152 at a first location 162 of the bar 152. The meshing of the gear teeth 142 with the teeth 154 of the bar 152 as the gear 136 rotates causes the bar 152 (and thus, the adjustable portion 104) to translate or move relative to the housing 102 to distract the adjustable implant 100. Further, this translation of the bar 152 causes the teeth 154 of the bar 152 at a second location 164 to mesh with the gear teeth 144 of the second gear assembly 134 which in turn positions the anvil 148 of the second gear assembly 134 such that the anvil 148 is in the rotating path of the protrusion 124 and will be engaged by the protrusion 124 during continued rotation of the actuator 112 as shown in FIG. 5.

Upon continued actuation of the actuator 112, the protrusion 124 is configured to engage with the anvil 148 of the second gear assembly 134. More specifically, upon actuation of the actuator 112, the impact hammer surface 126 (FIG. 3) of the protrusion 124 provides a striking force on the anvil 148 of the second gear assembly 134 as shown in FIG. 6. Such engagement and/or striking force causes the gear 138 to rotate and the gear teeth 144 to mesh with the teeth 154 on the bar 152 at a third location 166 of the bar 152. The meshing of the gear teeth 144 with the teeth 154 of the bar 152 as the gear 138 rotates causes the bar 152 (and thus, the adjustable portion 104) to translate or move relative to the housing 102 to distract the adjustable implant 100. Further this translation of the bar 152 causes the teeth 154 of the bar 152 at a fourth location 168 to mesh with the gear teeth 142 of the first gear assembly 132 which in turn positions the anvil 146 of the first gear assembly 132 such that the anvil 146 is in the rotating path of the protrusion 124 and will be engaged by the protrusion 124 during continued rotation of the actuator 112.

While the foregoing describes operation to distract the adjustable implant 100, in some applications, it may be desirable to retract or shorten the adjustable implant 100. The adjustable implant 100 as shown in FIG. 6 can be retracted by causing the actuator 112 to rotate in the opposite way of that described in FIGS. 4-5 such that each strike of the protrusion 124 about anvils 146, 148 causes the gears 136, 138 to rotate in the opposite direction such that as the gear teeth 142, 144 mesh with the bar 152, the bar 152 retracts and moves the adjustable portion 104 toward the housing 102. Retraction may be desirable for non-union fractures.

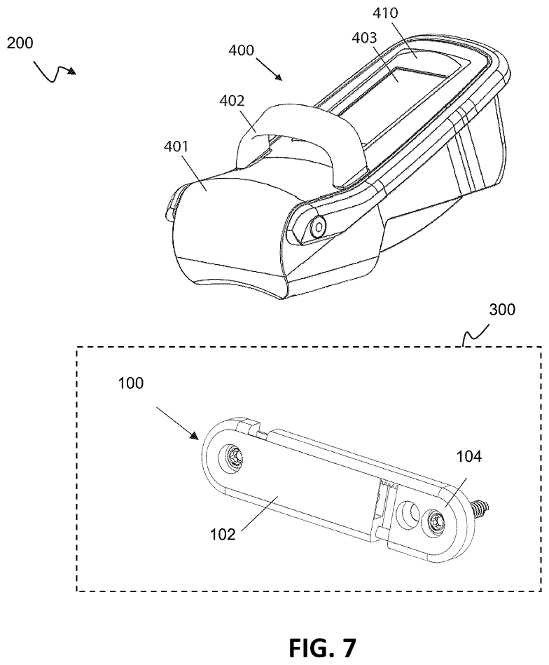

Actuation of the actuator 112 can be caused and controlled by an external adjustment device such as those described in Published International Application No. WO 2020/163800 A1, U.S. Pat. Nos. 8,382,756, 9,248,043, 9,078,711, 9,044,281, 11,246,694, U.S. Published Application No. US 2016/0113683 A1, and U.S. Pat. No. 10,835,290, all of which are incorporated herein by reference as if set forth in their entirety. Thus, the disclosure also relates to a system 200 (FIG. 7) for adjusting the position of two bone portions relative to each other. The system 200 can include the adjustable implant 100 fixed within a patient 300 and an external adjustment device 400 positioned external to the patient 300. The external adjustment device 400, may include a housing 401 having a handle 402 and a display 403. The handle 402 is shown extending upwardly from the housing 401. In some embodiments, the display 403 may be integrated with the housing 401 of the external adjustment device 400. In the illustrated embodiment, the external adjustment device 400 is configured to receive a removable controller 410 having a display 403, with the display 403 being an integral part of the removable controller 410.

According to an exemplary embodiment, the controller 410 may be a handheld electronic device. The handheld electronic device may be, for example, a smartphone, a tablet, and any other known handheld electronic device. The handheld electronic device may contain and may be operatively connected to a display and/or one or more wireless communication protocols (e.g., Wi-Fi or Bluetooth®). The display of the handheld electronic device may be disposed adjacent to a top surface of the external adjustment device 400, such that the display 403 can communicate information to and receive instructions from a user during use.

For example, in some embodiments the display 403 may present to a user a graphical user interface (GUI). The display 403 may include one or more of a touchscreen or touchscreen technology, including, for example, capacitive touchscreen technology. The GUI may communicate adjustment instructions to a user which may correspond to a treatment regimen to guide the user in adjusting the adjustable implant in accordance with the treatment regimen. Additionally, the GUI may include one or more touchscreen digital buttons configured to activate and control the external adjustment device 400.

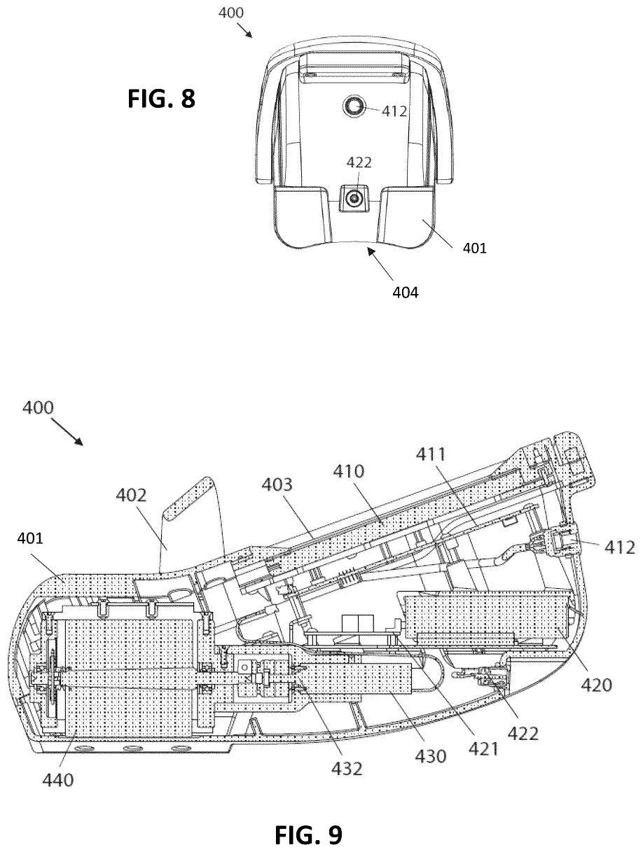

FIG. 8 shows a front view of the external adjustment device 400, the external adjustment device 400 including a power supply input 422 and a data connection port 412. Additionally, a bottom surface 404 of the housing 401 is shown including a curvature configured to form to a patient's body and minimize a distance (gap) between the magnet 440 (FIGS. 9-10 and 11A-11B) and a magnet 118 (FIG. 3) of the adjustable implant 100 (FIG. 7). The power supply input 422 may be configured to removably receive an AC power supply. The data connection port 412 may be configured to removably receive a data communication cable. The data communication cable may be configured to connect the external adjustment device 400 to a tertiary device to perform one or more of updating the controller 410 software and downloading data from the controller 410.

FIG. 9 shows a cross-sectional side view of the external adjustment device 400 in accordance with the first embodiment. The external adjustment device 400 is shown including the housing 401, the controller 410, an internal power storage device 420, a motor 430, and at least one magnet 440.

The internal power storage device 420 and wireless communication capabilities of the controller 440, may provide for wireless operation of the external adjustment device 400. The internal power storage device 420 may negate the need for a power cord during operation. The controller 410 may provide a low voltage control system negating the need for a bulky external control module. And wireless communication capabilities, for example one or more of radio frequency (RF), Wi-Fi, or Bluetooth® may enable the external adjustment device 400 and the controller 410 to operate remotely. The remote operation may be achieved by one or more of a tertiary device in the same room, and across the internet by a tertiary device on the other side of the globe.

In some embodiments, the controller 410 may be a control board disposed within the housing 401 of the external adjustment device 400. The display 403 may include any type of display 403, including for example: LED, LCD, OLED, and any other known display and touchscreen technology. The control interface board 411 may contain or be in communication with one or more communication circuit, for example, one or more of Wi-Fi, cellular networks, or Bluetooth®, enabling communication between the external adjustment device 400 and one or more tertiary devices.

In FIG. 9, the controller 410 is shown operably connected to a controller interface board 411 by at least one interconnect. In some embodiments, this connection may be established via a physical connection as illustrated, while in other embodiments this connection may be established via a wireless connection, for example, via Bluetooth®. The control interface board 411 may be further connected to one or more of a power interface board 421, the power storage device 420, and the actuator 430.

The controller 410 may be remotely accessible and remotely controllable by a tertiary device allowing for remote operation of the external adjustment device 400 by a user from outside of a sterile field.

The external adjustment device 400 is also shown including an internal power storage device 420. The power storage device 420 may include a battery, a capacitor, and any other power storage device known and used in the art. The power storage device may be rechargeable and the external adjustment device 400 may include a recharging circuit configured to recharge the power storage device 420 using an external power source. The external power source, for example a power supply, may be operably connected to the recharging circuit of the power storage device via the power supply input 422. The power storage device 420, and/or at least a portion of the recharging circuit, may be disposed adjacent to a surface of the external adjustment device 400, enabling connection of a power supply charge cable to the external adjustment device 400. In some embodiments, the recharging circuit may enable wireless charging of the internal power storage device 420, using induction to wirelessly transfer power. In some embodiments, the recharging circuit may be part of and connected to one or more of the power distribution board 421 and the power storage device 400.

In the illustrated embodiment, the power storage device 420 is a battery. The battery 420 is mounted to a chassis of the external adjustment device 400, adjacent to a surface of the external adjustment device 400 enabling connection of a power supply to the external adjustment device 400 at a power supply input 422. The battery 420 includes a power interface board 421, configured to interface with and communicate power to the motor 430. The power interface board 421 may be operably coupled to one or more of the motor 430 and the control interface board 411. The power interface board 421 may also communicate electrical energy from one or more of a power supply input 422 and the power storage device 420, to the controller 410.

The actuator of the external adjustment device 400 includes an electronic motor 430. The driver of the external adjustment device 400 includes a magnet 440 rotatably coupled to the electronic motor 430. The motor 430 may be operably connected to one or more of the controller 410, the control interface board 411, the power interface board 421, and the internal power storage device 420. In the illustrated embodiment the electronic motor 430 is operably connected to the internal power storage device 420 by the power interface board 421. The power interface board 421 may include power distribution circuits to communicate electrical energy to the electronic motor 430 from one or more of the power supply input 422 and the internal power storage device 420. The power interface board 421 may also be operably connected to the control interface board 411, to relay control information from the controller 410 to the motor 430. In some embodiments, the controller 410 may be in direct communication with the motor 430, and in some embodiments the controller 410 may be connected to the electronic motor via a wireless connection, for example a Bluetooth® connection.

The motor 430 may include any type of motor capable of rotating the magnet 440. The motor 430 is an electric motor and may include a rotational speed sensor 432. The rotational speed sensor 432 may be connected to and in communication with one or more of the control interface board 411 and the controller 410. In some embodiments, the internal speed sensor 432 may include for example one or more of an encoder and a digital output of an electronic motor. In some embodiments, the motor 430 is configured to communicate rotational speed data to the controller 410 wirelessly.

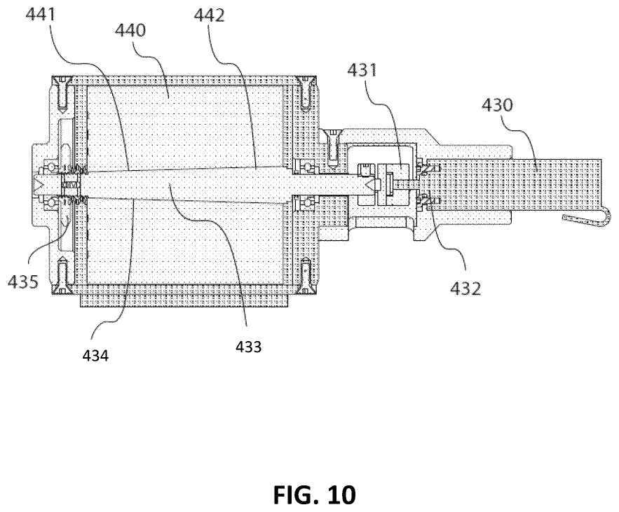

FIG. 10 shows an enhanced cross-sectional view of the motor 430 and the magnet 440 of the external adjustment device 400 (FIG. 9) in accordance with a first embodiment. The magnet 440 is shown rotatably coupled to the motor 430 by one or more couplings 431. In the illustrated embodiment, the magnet 440 includes an internal cavity 441 having an internal surface 442 and having a tapered profile. A magnet drive shaft 433 is shown including a magnet contact surface 434 having a tapered profile. The tapered profile of the magnet drive shaft 433 is configured to communicate with the tapered profile of the internal surface 442 of the magnet 440. This enables the magnet 440 to be secured to the magnet drive shaft 433 by a friction fit. The magnet 440 may be configured to be held onto the magnet drive shaft 433 by a cap 435 and the communicating tapered profiles. In some embodiments, the magnet 440 may be attached to the magnet drive shaft 433 using an adhesive material.

The magnet 440 may comprise any magnetic element including a radially polarized cylindrical magnet, a permanent magnet, an electromagnet, and any other magnetic element known and used in the art. The magnet 440 is configured to magnetically couple with a permanent magnet 118 (FIG. 3) of an adjustable implant 100 (FIG. 7) and to rotate the permanent magnet 118 and adjust the adjustable implant 100. Upon a rotation of the magnet 440, a rotating magnetic field will be generated, placing a force on the magnetically coupled permanent magnet 118 of the adjustable implant 100, thereby inducing a rotation of the permanent magnet 118 and subsequent adjustment of the adjustable implant 100.

In some embodiments, the external adjustment device 400 includes one or more sensors configured to monitor a rotational speed of the magnet 440. In some embodiments, the sensors include magnetic sensors, for example Hall-Effect sensors disposed on one or more of the housing 401 (FIG. 9), a plate, and a chassis, and may be placed adjacent to the magnet 440. In some embodiments, the sensors include photo-sensors. The magnet may include one or more circular optical encoder strips to work in conjunction with the photo-sensors. U.S. Published Application No. US 2016/0113683 A1, previously incorporated by reference, describes various systems and methods for non-invasively detecting the force generated by a non-invasively adjustable implant.

In the illustrated embodiment, the external adjustment device 400 includes a motor 430 having one or more rotational speed sensor 432 configured to detect a change in a motor angular velocity (V). As described below, sensor 432 may be used to non-invasively detect a rotation of the permanent magnet 118 of the adjustable implant 100. The motor 430 has torque characteristics that allow for little variation in motor angular velocity (V) during a motor rotation and corresponding magnet 440 rotation, when there is no implant or ferrous material located near the ERC magnet or magnetically coupled to the magnet 440.

When an adjustable implant 100 having a magnet 118 is in close proximity to the rotating magnet 440, and is for example magnetically coupled to the magnet 440, the magnetic poles of both magnets cause a changing load on the motor 430 twice per revolution. This causes the magnet 440 to increase or decrease in angular velocity, with the variations detectable by the rotational speed sensor 432.

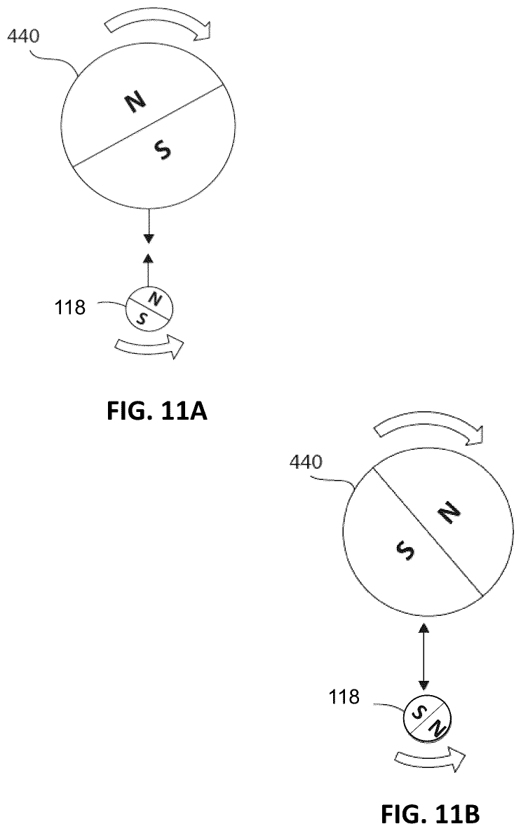

In FIG. 11A, the magnet 440 of the external adjustment device 400 is shown rotating in a first clockwise direction, with the magnet 118 of the adjustable implant 100 (FIG. 7) shown magnetically coupled to the magnet 440 and rotating in a second counterclockwise direction. As one with skill in the art may appreciate, as the motor 430 (FIG. 10) drives rotation of the magnet 440, the respective poles of the magnet 440 and the magnet 118 will attract each other, placing a reduced load on the motor 430 to drive the rotation as the poles are directed towards each other. Comparatively in FIG. 11B, as the motor 430 continues to drive rotation of the magnet 440 the respective poles of the magnet 440 and the permanent magnet 118 will still attract each other, placing an increased load on the motor 430 to drive the rotation as the poles are directed away from each other. These changes in load result in observable changes of the angular velocity that can be detected by the rotational speed sensor 432 of the motor 430. As it should be appreciated, the rotation of the magnet 440 in one direction results in distraction (or lengthening) of the adjustable implant 100 and rotation of the magnet 440 in the other direction results in retraction (or shortening) of the adjustable implant 100.

The magnet 440 may be configured to rotate at an exemplary speed of 200 revolutions per minute (RPM) or greater. This in turn may result in the magnet 118 having a relatively greater rotational speed in RPM, thereby causing the impact hammer force. Specifically, the magnet 118 can rotate at 700 RPM or greater to provide the impact hammer force. Referring to FIGS. 11A-11B, as the south pole of the magnet 440 rotates, the north pole of the magnet 118 of the adjustable implant 100 is attracted and pulled. As the south pole of the magnet 440 continues around, the magnet 118 of the adjustable implant 100 is bounced back by the impact reaction forces thereby readying the magnet 118 for another blow.

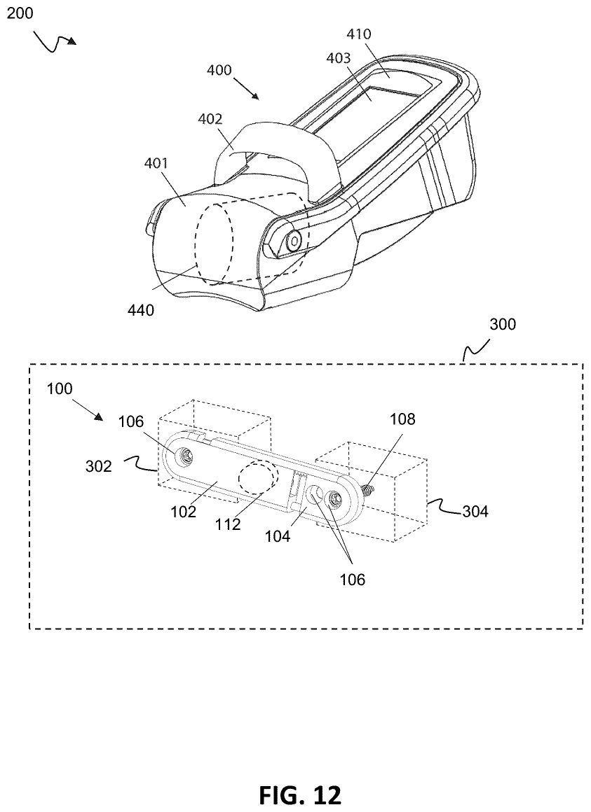

Aspects of the disclosure also include a method for non-invasively adjusting an adjustable implant 100. Referring to FIGS. 2-6 and 12, the method includes providing the adjustable implant 100 having a housing 102 configured to be coupled to a first bone portion 302 (FIG. 12), an adjustable portion 104 configured to be coupled to a second bone portion 304 (FIG. 12), an actuator 112 rotationally mounted within the housing 102 and including a protrusion 124 (FIGS. 2-6) extending therefrom, and at least one gear 136, 138 (FIGS. 2 and 4-6) having an anvil 146, 148 (FIGS. 2 and 4-6) coupled thereto. As shown in FIGS. 2 and 4-6, the adjustable portion 104 includes at least a first bar 152. The method also includes coupling and/or fixing the housing 102 to the first bone portion 302 and coupling and/or fixing the adjustable portion 104 to the second bone portion 304. The housing 102 and adjustable portion 104 can be coupled/fixed to the respective bone portions 302, 304 via bone anchors 108 (FIGS. 1-2 and 12) inserted within fixation apertures 106 (FIGS. 1-2) using known techniques such as via creating an operative corridor (e.g., to access the spine, intramedullary canal, or extramedullary location), optionally creating an osteotomy, and driving the bone anchors 108 through the fixation apertures 106 using a driver (not shown). Further, the method includes non-invasively adjusting the adjustable implant 100 by causing rotation of the actuator 112 such that the protrusion 124 (FIGS. 2 and 4-6) of the actuator 112 engages with the anvil 146, 148 during rotation of the actuator 112 to cause the adjustable portion 104 to move relative to the housing 102.

More specifically, the at least one gear can include a first gear 136 having a first anvil 146 coupled thereto and a second gear 138 having a second anvil 148 coupled thereto. During rotation of the actuator 112, the protrusion 124 engages the first anvil 146 to cause the first gear 136 to interact with the first bar 152 at a first location 162 (FIG. 4) on the first bar 152 to cause the adjustable portion 104 to move relative to the housing 102. The interaction of the first gear 138 with the first bar 152 causes the first bar 152 to interact with the second gear 138 at a second location 164 (FIG. 4) on the first bar 152. The interaction of the first bar 152 with the second gear 138 at the second location 164 causes the second anvil 148 to be positioned such that the second anvil 148 shall be engaged by the protrusion 124 during another revolution of the actuator 112 upon continued rotation or due to another treatment session.

After the protrusion 124 engages the first anvil 146, the protrusion 124 engages the second anvil 148 as shown in FIG. 5 to cause the second gear 138 to interact with the first bar 152 to cause the adjustable portion 104 to move relative to the housing 102. The interaction of the second gear 138 with the first bar 152 causes the first bar 152 to interact with the first gear 136 at a third location 168 (FIG. 6) of the first bar 152. The interaction of the first bar 152 with the first gear 136 at the third location 168 causes the first anvil 146 to be positioned such that the first anvil 146 shall be engaged by the protrusion 124 during continued revolution of the actuator 112.

The external adjustment device 400 (FIGS. 7-10 and 12) can be used to non-invasively adjust the adjustable implant 100. Specifically, the external adjustment device 400 can be positioned perpendicularly to the adjustable implant, i.e., the rotating magnet 440 of the external adjustment device 400 can be positioned such that the axis of rotation is perpendicular to the axis of rotation of magnet 118 of the adjustable implant 100. As described herein, the external adjustment device 400 can be positioned against the patient's skin or clothes in proximity to the adjustable implant 100 to provide a magnetic field to the actuator 112 to cause rotation of the actuator 112. As the actuator 112 rotates, the protrusion 124 having an impact hammer surface 126 (FIG. 3) can strike the anvils 146, 148 during rotation to cause translation of the first bar 152, and therefore, the adjustable portion 104 relative to the housing 102. This in turn causes movement of the second bone portion 304 (FIG. 12) relative to the first bone portion 302 (FIG. 12). The impact drive of the adjustable implant 100 provides advantages over direct drive adjustable implants by providing improved force characteristics. The magnet 440 of the external adjustment device 400 is configured to rotate at 200 revolutions per minute (RPM) or greater which in turn results in the actuator 112 having a relatively greater rotational speed in RPM, thereby causing the impact hammer force. Specifically, the actuator 112 can rotate at 700 RPM or greater to provide the impact hammer force. Referring back to FIGS. 11A-11B, as the north pole of the magnet 440 rotates, the north pole of the magnet 118 of the adjustable implant 100 is repelled. As the south pole of the magnet 440 continues around, the magnet 118 of the adjustable implant 100 is pulled back causing a bounce back, thereby readying the magnet 118 for another blow.

Distraction of the adjustable implant 100 can be performed at a desirable rate and duration depending on the desired treatment plan of a medical professional for a particular patient or treatment application. Distraction of the adjustable implant 100 is used to facilitate and control growth of new bone between the two bone portions 302, 304. Retraction of the adjustable implant 100 can be performed in the case of non-union fractures.

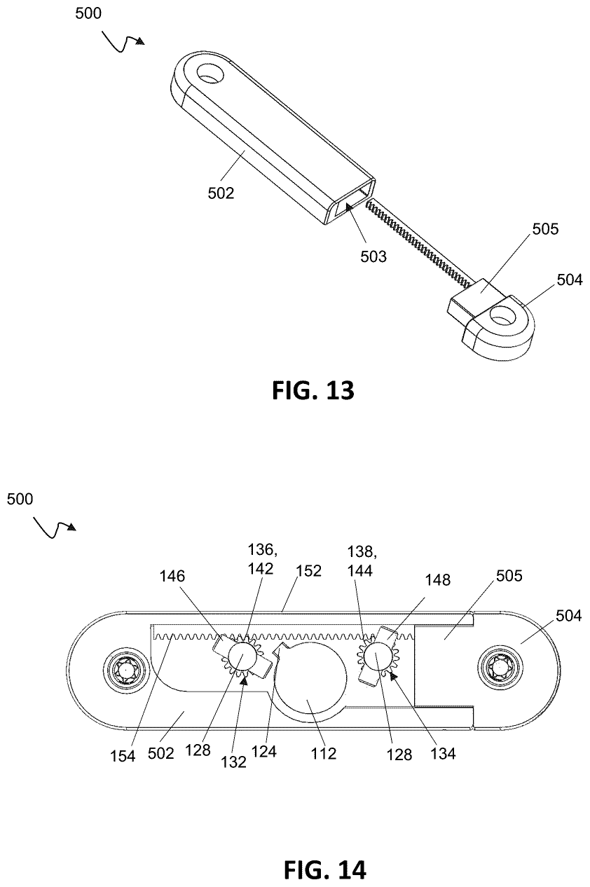

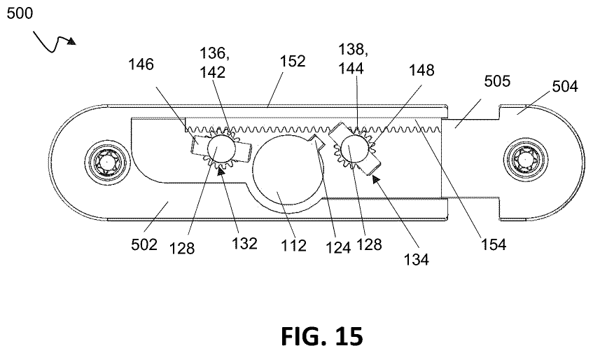

FIGS. 13-14 show an adjustable implant 500 according to another embodiment of the disclosure. In this embodiment, the adjustable implant 500 includes a housing 502 having a cavity 503 (FIG. 13) therein. In this embodiment, the cavity 503 is completely sealed by the housing 502 and a boss 505 of an adjustable portion 504 positioned within the housing 502. The boss 505 can be integrally formed as part of the adjustable portion 504 with the first bar 152. Alternatively, the boss 505 can be a separate component fixed to the adjustable portion 504 and the first bar 152. The boss 505 can be of any complementary shape and/or dimension of the cavity 503 and/or housing 502 such that the cavity 503 remains sealed even during adjustment of the adjustable implant 500. To further aid in scaling, the boss 505 can include a groove in which an o-ring may be positioned and disposed within the groove between the boss 505 and the housing 502. Additionally, the boss 505 can further aid in providing strength and alignment to the adjustable portion 504 as the adjustable portion moves relative to the housing 502, thus, the second bar 156 (FIGS. 2 and 4-6) is not required in this embodiment. The first bar 152 can be wholly positioned within the cavity 503 within the housing 502 in the fully retracted position (FIG. 14) and within a retracted position (FIG. 15). The remainder of the components within the adjustable implant 500 are similar to that of adjustable implant 100 (FIGS. 1-6). Thus, like numbering has been used and duplicate description has been omitted for brevity. Further, the adjustable implant 500 operates in the same way as the adjustable implant 100 (e.g., by non-invasively adjusting the adjustable implant 500 with external adjustment device 400 (FIGS. 7-12)).

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The terminology used herein is for the purpose of describing particular embodiments only and is not intended to be limiting of the disclosure. As used herein, the terms “first,” “second,” and the like, do not denote any order, quantity, or importance, but rather are used to distinguish one element from another, and the terms “a” and “an” herein do not denote a limitation of quantity, but rather denote the presence of at least one of the referenced item. It will be further understood that the terms “comprises” and/or comprising,” when used in this specification, specify the presence of stated features, integers, steps, operations, elements, and/or components, but do not preclude the presence or addition of one or more other features, integers, steps, operations, elements, components, and/or groups. As used herein, “substantially” refers to largely, for the most part, entirely specified or any slight deviation which provides the same technical benefits of the disclosure.

The implementations set forth in the foregoing description do not represent all implementations consistent with the subject matter described herein. Instead, they are merely some examples consistent with aspects related to the described subject matter. Although a few variations have been described in detail herein, other modifications or additions are possible. In particular, further features and/or variations can be provided in addition to those set forth herein. For example, the implementations described above can be directed to various combinations and sub-combinations of the disclosed features and/or combinations and sub-combinations of one or more features further to those disclosed herein. In addition, the logic flows depicted in the accompanying figures and/or described herein do not necessarily require the particular order shown, or sequential order, to achieve desirable results. The scope of the following claims may include other implementations or embodiments.

What is claimed is:

1. An adjustable implant comprising:
a housing configured to be coupled to a first bone portion;
an adjustable portion configured to be coupled to a second bone portion, the adjustable portion having a first bar;
an actuator rotationally mounted within the housing, the actuator including a protrusion extending therefrom; and
at least one gear having an anvil coupled thereto, wherein the protrusion of the actuator is configured to engage the anvil during rotation of the actuator to cause the adjustable portion to move relative to the housing.

2. The adjustable implant of claim 1, wherein the protrusion includes an impact hammer surface.

3. The adjustable implant of claim 1, wherein the first bar includes a gear rack.

4. The adjustable implant of claim 3, wherein the gear rack includes a plurality of teeth for meshing with the at least one gear thereby causing the adjustable portion to move relative to the housing.

5. The adjustable implant of claim 1, wherein the adjustable portion includes a second bar at least partially disposed within the housing and configured to move relative to the housing as the adjustable portion moves relative to the housing.

6. The adjustable implant of claim 1, wherein the at least one gear includes a pinion gear.

7. The adjustable implant of claim 1, wherein the at least one gear includes a first gear having a first anvil and a second gear having a second anvil.

8. The adjustable implant of claim 7, wherein the first gear and the second gear are each disposed within the housing on opposing sides of the actuator.

9. The adjustable implant of claim 7, wherein the protrusion of the actuator is configured to engage the first anvil of the first gear and the second anvil of the second gear during rotation of the actuator, whereby engagement of the first and second anvils by the protrusion causes the first and second gears to interact with the first bar thereby causing the adjustable portion to move relative to the housing.

10. The adjustable implant of claim 9, wherein the engagement of the protrusion with the second anvil during rotation of the actuator causes the first bar to interact with the first gear to cause the first anvil to be positioned such that, upon continued rotation of the actuator, the protrusion of the actuator engages the first anvil.

11. The adjustable implant of claim 9, wherein the engagement of the protrusion with the first anvil during rotation of the actuator causes the first bar to interact with the second gear to cause the second anvil to be positioned such that, upon continued rotation of the actuator, the protrusion of the actuator engages the second anvil.

12. The adjustable implant of claim 1, wherein the actuator includes a magnet disposed within a magnet casing, wherein the protrusion extends from the magnet casing.

13. The adjustable implant of claim 12, wherein the magnet is configured to rotate by application of a non-invasively applied magnetic field.

14. The adjustable implant of claim 1, wherein the housing and the adjustable portion each include an aperture for accommodating a fixation screw therein.

15. An adjustable implant comprising:
a housing configured to be coupled to a first bone portion;
an adjustable portion configured to be coupled to a second bone portion, the adjustable portion having a toothed rack;
a magnet assembly rotationally mounted within the housing, the magnet assembly including a protrusion extending therefrom, the protrusion having an impact hammer surface; and
at least one gear having an anvil coupled thereto, wherein the protrusion of the magnet assembly is configured to engage the anvil during rotation of the magnet assembly, whereby such engagement of the anvil by the impact hammer surface of the protrusion causes the at least one gear to interact with the toothed rack thereby causing the adjustable portion to move relative to the housing.

16. An adjustable implant comprising:
a housing configured to be coupled to a first bone portion;
an adjustable portion configured to be coupled to a second bone portion, the adjustable portion having a toothed rack;
a magnet assembly rotationally mounted within the housing, the magnet assembly including a protrusion extending therefrom;
a first gear having a first anvil coupled thereto; and
a second gear having a second anvil coupled thereto, wherein the protrusion of the magnet assembly is configured to engage the first and second anvils during rotation of the magnet assembly, whereby such engagement of the first and second anvils by the protrusion causes the first and second gears to interact with the toothed rack thereby causing the adjustable portion to move relative to the housing.

17. A method of non-invasively adjusting an adjustable implant, the method comprising:
providing an adjustable implant, the adjustable implant including:
a housing configured to be coupled to a first bone portion;
an adjustable portion configured to be coupled to a second bone portion, the adjustable portion having a first bar;
an actuator rotationally mounted within the housing, the actuator including a protrusion extending therefrom; and
at least one gear having an anvil coupled thereto;
coupling the housing to the first bone portion;
coupling the adjustable portion to the second bone portion; and
non-invasively adjusting the adjustable implant by causing rotation of the actuator such that the protrusion of the actuator engages with the anvil during rotation of the actuator to cause the adjustable portion to move relative to the housing.

18. The method of claim 17, wherein the at least one gear includes a first gear having a first anvil coupled thereto and a second gear having a second anvil coupled thereto.

19. The method of claim 18, wherein, during the rotation of the actuator, the protrusion engages the first anvil to cause the first gear to interact with the first bar at a first location of the first bar to cause the adjustable portion to move relative to the housing.

20. The method of claim 19, wherein the interaction of the first gear with the first bar causes the first bar to interact with the second gear at a second location of the first bar.

21. The method of claim 20, wherein the interaction of the first bar with the second gear at the second location causes the second anvil to be positioned such that the second anvil shall be engaged by the protrusion during another revolution of the actuator.

22. The method of claim 20, wherein, after the protrusion engages the first anvil, the protrusion engages the second anvil to cause the second gear to interact with the first bar to cause the adjustable portion to move relative to the housing.

23. The method of claim 22, wherein the interaction of the second gear with the first bar causes the first bar to interact with the first gear at a third location of the first bar.

24. The method of claim 23, wherein the interaction of the first bar with the first gear at the third location causes the first anvil to be positioned such that the first anvil shall be engaged by the protrusion during another revolution of the actuator.

25. A system comprising:
an adjustable implant including:
a housing configured to be coupled to a first bone portion;
an adjustable portion configured to be coupled to a second bone portion, the adjustable portion having a first bar;
an actuator rotationally mounted within the housing, the actuator including a protrusion extending therefrom; and
at least one gear having an anvil coupled thereto, wherein the protrusion of the actuator is configured to engage the anvil during rotation of the actuator to cause the adjustable portion to move relative to the housing; and
an external adjustment device configured to cause actuation of the actuator upon being positioned such that the external adjustment device is perpendicular to the actuator.

26. The system of claim 25, wherein the actuator includes a magnet disposed within a magnet casing, wherein the protrusion extends from the magnet casing.

27. The system of claim 26, wherein the magnet is configured to rotate by application of a non-invasively applied magnetic field from the external adjustment device.

28. The system of claim 26, wherein the external adjustment device comprises at least one rotating magnet, wherein the rotating magnet of the external adjustment device is positioned perpendicular to the magnet of the actuator thereby causing actuation of the actuator.

29. The system of claim 26, wherein the protrusion includes an impact hammer surface.

30. The system of claim 29, wherein the impact hammer surface includes chrome plating.

31. The system of claim 25, wherein the first bar includes a gear rack.

32. The system of claim 31, wherein the gear rack includes a plurality of teeth configured to mesh with the at least one gear, thereby causing the adjustable portion to move relative to the housing.

33. The system of claim 25, wherein the adjustable portion includes a second bar at least partially disposed within the housing and configured to move relative to the housing as the adjustable portion moves relative to the housing.

34. The system of claim 25, wherein the at least one gear includes a pinion gear.

35. The system of claim 25, wherein the at least one gear includes a first gear having a first anvil coupled thereto and a second gear having a second anvil coupled thereto.

36. The system of claim 35, wherein the first gear and the second gear are each disposed within the housing on opposing sides of the actuator.

37. The system of claim 35, wherein the protrusion of the actuator is configured to engage the first anvil of the first gear and the second anvil of the second gear during rotation of the actuator, whereby engagement of the first and second anvils by the protrusion causes the first and second gears to interact with the first bar thereby causing the adjustable portion to move relative to the housing.

38. The system of claim 37, wherein the engagement of the protrusion with the second anvil during rotation of the actuator causes the first bar to interact with the first gear to cause the first anvil to be positioned such that, upon continued rotation of the actuator, the protrusion of the actuator engages the first anvil.

39. The system of claim 37, wherein the engagement of the protrusion with the first anvil during rotation of the actuator causes the first bar to interact with the second gear to cause the second anvil to be positioned such that, upon continued rotation of the actuator, the protrusion of the actuator engages the second anvil.

40. The system of claim 25, wherein the housing and the adjustable portion each include an aperture for accommodating a fixation screw therein.

41. An assembly for an adjustable implant, the assembly comprising:
an actuator rotatably mounted within the adjustable implant, the actuator including a protrusion extending therefrom;
a first gear having a first anvil coupled thereto; and
a second gear having a second anvil coupled thereto,
wherein rotation of the actuator causes the protrusion to engage the first anvil and the second anvil to cause the adjustable implant to adjust.

42. The assembly of claim 41, wherein the protrusion includes an impact hammer surface for striking each of the first anvil and the second anvil to cause the adjustable implant to adjust.

43. The assembly of claim 41, wherein the engagement of the protrusion with the first anvil causes the second anvil to be positioned in such a way that, upon continued rotation of the actuator, the second anvil is to be engaged by the protrusion.

44. The assembly of claim 41, wherein the first gear and the second gear are positioned on opposing sides of the actuator within the adjustable implant.

45. The assembly of claim 41, wherein the adjustable implant includes a housing and an adjustable portion, wherein the actuator is rotatably mounted within the housing.

46. The assembly of claim 45, wherein the first gear and second gear each have gear teeth configured to mesh with teeth positioned on the adjustable portion.

47. The assembly of claim 46, wherein the engagement of the first anvil with the protrusion causes the first gear to rotate thereby causing the adjustable portion to move relative to the housing.

48. The assembly of claim 47, wherein the engagement of the second anvil with the protrusion causes the second gear to rotate thereby causing the adjustable portion to move relative to the housing.
